(19) United States
(12) Patent Application Publication
(10) Pub. No.: US 2026/0263787 A1
(43) Pub. Date: Sep. 10, 2026

(54) DIFFUSION PROMOTION DEVICE AND ION INTRODUCTION SYSTEM

(71) Applicant: CYSAY INC., Tokyo (JP)

(72) Inventors: Yasuhiro Yamashita, Toyko (JP); Bungo Watanabe, Kawasaki-shi (JP)

(73) Assignee: CYSAY INC., Tokyo (JP)

(21) Appl. No.: 19/644,300

(22) Filed: Apr. 10, 2026

Related U.S. Application Data
(63) Continuation of application No. PCT/JP2024/035917, filed on Oct. 8, 2024.

(30) Foreign Application Priority Data
Oct. 10, 2023 (JP) 2023-175352

(51) Int. Cl. A61N 1/30; A61N 1/04

(52) U.S. Cl. CPC A61N 1/303; A61N 1/0448

(57) ABSTRACT

The purpose of the invention is to promote diffusion of an active ingredient contained in a supplied liquid agent under a surface layer, using a compact configuration. A diffusion promotion device comprises a first cylindrical member and a second cylindrical member. In a state in which a liquid agent has been or is being supplied to an object from a liquid agent supply unit of a liquid agent supply device accommodated in an internal accommodation space of the diffusion promotion device, when a current path is formed between a first electrode formed on a side surface of the first cylindrical member and a second electrode formed on a tip end portion of the second cylindrical member through a surface layer portion of the object, diffusion of the active ingredient contained in the liquid agent under the surface layer of the object (for example, the surface of the skin) is promoted is promoted.

### TECHNICAL FIELD

The present invention relates to a diffusion promotion device and an ion introduction system.

### BACKGROUND ART

Traditionally, pharmaceuticals and other substances have been widely administered in a transdermal route. A technique by using minute needles called “microneedles” has been proposed for such transdermal administration as one technique. In the technique, a fixed amount of liquid agent is ejected through hollow microneedles in conjunction with the operation of a switch mechanism, such as a click-action type (see Patent Document 1; hereinafter referred to as “prior art 1”). According to the technique disclosed in the prior art 1, liquid agents can be delivered to the desired location on a surface layer of an epithelium, such as a skin surface or a stratum corneum, through simple operation.

Furthermore, a technique for an adhesive patch for epithelium has been proposed (see Patent Document 2; hereinafter referred to as “prior art 2”). The adhesive patch has multiple microneedles composed of a water-soluble polymer that dissolves in vivo and an active ingredient, which is a physiologically active substance. In the prior art 2, the patch is adhered on the epithelium surface for administering the physiologically active substance by dissolving the microneedles.

### PRIOR ART DOCUMENTS

### Patent Documents

Patent Document 1: JP2023-094304A

Patent Document 2: JP6505210B

### SUMMARY OF INVENTION

### Problems to be Solved by the Invention

In the prior arts 1 and 2, the administered active ingredient to the object is diffused under the epithelium relying on the inherent mechanisms thereof. Therefore, there was room for the diffusion of the active ingredient under the epithelium from the viewpoint of technical improvement.

Furthermore, the technology disclosed in the prior art, the liquid agent dispensed through the microneedles may overflow onto the epithelial surface. Most of the overflowed liquid agent is released into the atmosphere due to drying or the like. Therefore, there was room for the efficient diffusion of the active ingredient under the epithelium from the perspective of the technical improvement.

When applying a liquid agent containing the active ingredient onto the epithelial surface being diffused relying on the object's own percutaneous mechanism, the same technical improvements are required.

The present invention was conceived in light of the circumstances described above. The present invention provides a diffusion promotion device and an ion introduction system that promotes the diffusion of an active ingredient contained in the administrated liquid agent under a surface layer of skin, having a compact configuration.

### Means for Solving the Problems

A diffusion promotion device according to first aspect of the present invention having an accommodation space for accommodating a liquid agent supply device for supplying the liquid agent into surface layer of an object is formed internally, which generates an electric current for promoting diffusion of an active ingredient in the liquid agent under a surface of an object, wherein the liquid agent supply device comprises: a shaft portion in which a liquid agent storage space for storing the liquid agent is formed along with a first direction; and a liquid agent supply unit disposed at a first end portion in the first direction side of the shaft portion, supplying the liquid agent contained in the liquid agent storage space to the object, and the diffusion promotion device comprises: a first cylindrical member having a first accommodation space being formed internally for accommodating a predetermined portion located in a second direction side, which is opposite to the first direction side, of the liquid agent supply device, and the first accommodation space is disposed along a third direction parallel to the first direction a second cylindrical member having a first through-hole and a second accommodation space being formed internally for accommodating a predetermined portion in the first direction side of the liquid agent supply device and being disposed in the third direction side of the first cylindrical member, wherein the second cylindrical member allows the liquid agent supply device along with the third direction so as to pass the first through-hole, and the liquid agent supply device along with the third direction not so as to pass the first through-hall, and; a first electrode being used to promote diffusion of the active ingredient; a second electrode being used to form a current path passing through the surface layer of the object with the first electrode when diffusion of the active ingredient is being promoted; and a battery unit that generates a predetermined potential difference between the first electrode and the second electrode, and wherein the accommodation space is formed of the first accommodation space and the second accommodation space.

In the first aspect, under the surface layer of the object, the diffusion of the active ingredient contained in the liquid agent may be promoted, when the current path is formed while the liquid agent is being supplied or has been supplied to the object from the liquid agent supply unit of the liquid agent supply device accommodated in the internal accommodation space. The current path may be formed between the first electrode and the second electrode, passing through the surface layer of the epithelium of the object. The ion introduction effect may promote the diffusion of the liquid agent under the surface layer of the object. As for the liquid agent supply unit, for example, the liquid applicator disclosed in prior art 1 may be suitably adopted.

In the first aspect, the diffusion promotion device having a compact configuration is capable of promoting the diffusion of the active ingredient contained in the supplied liquid agent under the surface layer of the object.

In the first aspect, the liquid agent supply unit is capable of including the liquid agent supply unit includes a plurality of microneedles disposed at the end portion in the first direction side, each microneedle being hollow to communicate with the liquid agent storage space; and the diffusion promotion device utilizes the plurality of microneedles to promote diffusion of the active ingredient being contained in the liquid agent supplied into the surface layer of the object. Even if the liquid agent supplied to the surface layer of the object through the microneedles overflows on the surface layer, the active ingredient contained in the overflowing liquid agent can be diffused under the surface layer of the object by the ion introduction effect, when the object can undergo a trans-surface absorption, such as in the case of the epidermis.

When the microneedles are disposed in the liquid agent supply unit, the liquid agent supply device includes: a first pressing operation member disposed at the end portion in the second direction side; and a liquid agent extrusion unit disposed in the shaft portion, which pushes out the liquid agent in the liquid agent storage space toward the liquid agent supply unit responding to pressure given to the first pressing operation member in the first direction, and wherein the microneedle is protruded outside the second cylindrical member via the first through-hole in response to the pressure given to the first pressing operation member in the first direction, and then it provides the liquid agent to the object through the tip thereof. The liquid agent is supplied to the object by being discharged from the tip of the microneedle that protrudes outside the second cylindrical member via the first through-hole in response to pressing the first pressing operation member in the first direction. Due to the press of the liquid agent supply unit against the surface layer of the object while pressing the first pressing operation member in the first direction, the liquid agent can be supplied from the tip of the microneedles protruding outside the second cylindrical member, via the first through-hole, to the surface layer of the object.

Here, the diffusion promotion device is capable of having a second through-hole being formed at the end portion in a fourth direction side of the first cylindrical member through which the shaft portion passes in the third direction, the fourth direction being opposite to the third direction, and further comprising; a second pressing operation member that is insertable into and removable from the internal space of the first cylindrical member via the second through-hole that transmits a pressing force corresponding to the pressing operation of the second pressing operation member in the third direction to the first pressing operation member of the liquid agent supply device accommodated in the accommodation space. When the pressure is applied to the second pressing operation member, a corresponding pressure force is transmitted to the first pressing operation member. As a result, the liquid agent is supplied to the surface layer of the object from the tip of the microneedles that protrudes outside the second cylindrical member via the first through-hole. Therefore, through the simple pressing operation on the second pressing operation member, the liquid agent can be supplied to the surface layer of the object from the tip of the microneedles protruding outside the second cylindrical member via the first through-hole.

The diffusion promotion device is capable of further comprising an elastic member, being capable of expanding and contracting in the fourth direction disposed in the second accommodation space, which biases the liquid agent supply device accommodated in the diffusion promotion device in the fourth direction, wherein the elastic member applies a biasing force to accommodate the entire liquid agent supply device into the accommodation space when the liquid agent is not supplied to the object. The elastic member applies a biasing force to accommodate the entire liquid agent supply device in the accommodation space when the liquid agent is not being supplied to the object. Therefore, when the liquid agent is not being supplied to the object, the end portion in the first direction side of the liquid agent supply unit of the liquid agent supply device is protected without requiring any special operation.

In the first aspect of the present invention, the liquid agent supply unit is capable to include a porous member absorbing and retaining the liquid agent, and the unit is disposed at the end portion in the first direction side and conducting, wherein the porous member provides the liquid agent to the object when it is pressed against the surface of the object, and wherein the diffusion promotion device promotes the diffusion of the active ingredient contained in the liquid agent provided by pressing the porous member against to the surface of the object under the surface layer of the object. The liquid agent is supplied to the surface of the object through a simple operation of pressing the porous material saturated with the liquid agent against the object. The ion introduction effect promotes the diffusion of the active ingredient under the surface of the object.

In the first aspect of the present invention, the first electrode is capable to be formed on at least a portion of the surface of the side of the first cylindrical member. Moreover, the second electrode is capable to be formed on at least a portion of the outer surface of the end portion in the third direction side of the second cylindrical member. For example, when a person includes an administration target, the following operation promotes diffusion of the active ingredient under the epithelium at the administration site. The operation is as follows: namely, the person grasps the side of the diffusion-promoting device with the palm and at least one finger (e.g., four fingers excluding the thumb) or multiple fingers (e.g., the thumb and at least one finger other than the finger to press the second pressing operation member) to contact the first electrode while the second electrode contacts the epithelial surface at the administration site.

In the first aspect of the present invention, the second cylindrical member is able to be detachably connected to the end portion in the third direction side of the first cylindrical member. The liquid agent supply device is accommodated in the accommodation space through the following procedure. That is, the liquid agent supply device is first inserted into the first accommodation space from the end portion in the third direction side of the first cylindrical member, while the first cylindrical member and the second cylindrical member are separated from each other. Subsequently, the liquid agent supply device is accommodated in the accommodation space due to the connection of the first cylindrical member with the second cylindrical member.

An ion introduction system according to second aspect of the present invention comprises: the diffusion promotion device according to claim 1; and the liquid agent supply device, wherein the liquid agent supply device includes: the shaft portion having the liquid agent storage space to store the liquid agent being formed in the first direction; and the liquid agent supply unit being disposed at the first end portion in the first direction side of the shaft portion for supplying the liquid agent stored in the liquid agent storage space to the object.

In the second aspect, a current path is formed between the first electrode and the second electrode, passing through the surface layer of the object while the liquid agent is supplied or has been supplied to the object from the liquid agent supply unit of the liquid agent supply device accommodated in the internal accommodation space of the diffusion promotion device; and the current path brings the ion introduction effect to promote the diffusion of the active ingredient contained in the supplied liquid agent under the surface layer of the object. Concurrently, the ion introduction system of the present invention in a compact configuration supplies the liquid agent containing active ingredient to the object and promotes the diffusion of the active ingredient contained in the supplied liquid agent under the surface layer of the object.

### Advantageous Effects of Invention

According to the diffusion promotion device of the present invention, it has a compact configuration, which promotes diffusion of the active ingredient contained in the liquid agent supplied to the target object under the surface of the object. According to the ion introduction device of the present invention, it supplies the liquid agent containing the active ingredient to the target object and promotes the diffusion of the active ingredient contained in the supplied liquid agent under the surface of the target object, using a compact configuration.

### BRIEF DESCRIPTION OF DRAWINGS

FIG. 1 is a perspective view illustrating the configuration of an ion introduction system according to an embodiment of the present invention.

FIG. 2 is a perspective view illustrating the configuration of the liquid agent supply device illustrated in FIG. 1.

FIG. 3 is a cross-sectional view (XZ sectional view) illustrating the portion positioned in the +Z direction (tip side) of internal configuration of the liquid agent supply device illustrated in FIG. 2.

FIGS. 4A, 4B and 4C show diagrams illustrating the change in the positional relationship between elements of the liquid agent supply device due to a pressing operation applied to the liquid agent supply device illustrated in FIG. 2.

FIG. 5 is a cross-sectional view (No. 1, XZ sectional view) illustrating the internal configuration of the diffusion promotion device illustrated in FIG. 1.

FIG. 6 is a cross-sectional view (No. 2, YZ sectional view) illustrating the internal configuration of the diffusion promotion device illustrated in FIG. 1.

FIGS. 7A, 7B and 7C show are diagrams illustrating the change in the positional relationship between the diffusion promotion device and the liquid agent supply device due to a pressing operation on the diffusion promotion device illustrated in FIG. 1.

### MODE FOR CARRYING OUT THE INVENTION

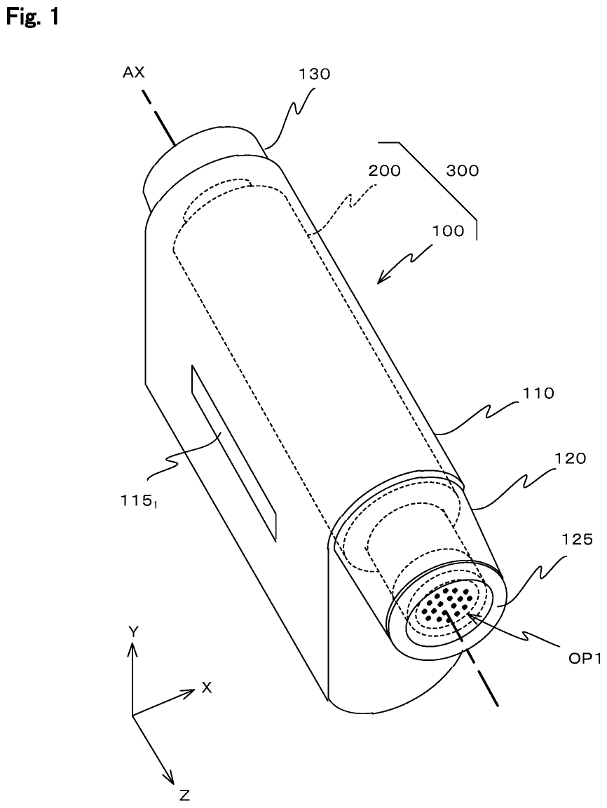

An embodiment of the present invention will be described with reference to FIG. 1 to FIG. 7C. In the following description and drawings, identical elements are designated by identical reference numerals, and redundant descriptions are omitted. Furthermore, in all drawings except FIG. 6, the dimensions and aspect ratios of each element are appropriately varied for clarity.

### Configuration

FIG. 1 illustrates a schematic configuration of an ion introduction system 300 according to the embodiment of the present invention, as viewed in perspective. As illustrated in FIG. 1, the ion introduction system 300 includes a diffusion promotion device 100 and a liquid agent supply device 200. The ion introduction system 300 of the embodiment is a system for performing ion introduction of active ingredient contained in a liquid agent, with a human being as a target object. The diffusion promotion device 100 is an embodiment of the diffusion promotion device according to the present invention.

As illustrated in FIG. 1, the diffusion promotion device 100 includes the first cylindrical member 110, the second cylindrical member 120 at the side in the +Z direction of the first cylindrical member 110, and a pressing member (the second pressing operation member) 130 at the side in the-Z direction of the first cylindrical member 110. The liquid agent supply device 200 may be accommodated in the internal accommodation space of the diffusion promotion device 100 formed with the first cylindrical member 110 and the second cylindrical member 120. As illustrated in FIG. 1, the +Z direction refers to the direction from the pressing member 130 toward the second cylindrical member 120. The −Z direction refers to the direction from the second cylindrical member 120 toward the pressing member 130. The +Z direction includes the first direction and a third direction that is parallel to the first direction. The −Z direction includes a second direction and the fourth direction that is parallel to the second direction.

The second cylindrical member 120 is detachably connected to the first cylindrical member 110. To enable this detachable connection, for example, a portion of the inner surface of the end portion in the +Z direction side (that is, the end portion sided in the +Z direction) of the first cylindrical member 110 and the outer surface of the end portion in the −Z direction side (that is, the end portion sided in the −Z direction) of the second cylindrical member 120 are formed with mating female and male threads.

Note that the diffusion promotion device 100 is sized to be easily grasped by a person's palm and at least one finger (e.g., four fingers excluding the thumb), or by multiple fingers (e.g., the thumb and at least one finger other than the finger(s) applying pressure to the pressing member 130).

As illustrated in FIG. 1, the first electrode 1151 is formed on the side surface sided in the −X direction of the first cylindrical member 110. Furthermore, although not illustrated in FIG. 1, the first electrode 115₂ is formed on the side surface sided in the +X direction of the first cylindrical member 110. Note that, when promoting the diffusion of the active ingredient of the liquid agent described later under an epithelium, at least a portion of the area gripping the diffusion promotion device 100 is brought into contact with at least one of the first electrode 115₁ and the first electrode 115₂.

Furthermore, as illustrated in FIG. 1, the first through-hole OP1 is formed at the end portion in the +Z direction side (that is, at the end portion sided in the +Z direction) of the second cylindrical member 120. A second electrode 125 is formed around the first through-hole OP1 at the end portion in the +Z direction side (that is, at the end portion sided in the +Z direction) of the second cylindrical member 120.

Here, to promote diffusion of the liquid agent's active ingredient under the epithelium, the surface sided in the +Z direction of the second electrode 125 is pressed against the epithelial surface at the liquid agent injection site. Therefore, during this diffusion promotion, a weak current path is formed between at least one of the first electrodes 115₁ and 115₂ and the second electrode 125 through the subepidermal layer.

The first cylindrical member 110 and the second cylindrical member 120 are made from insulating materials. Examples of these insulating materials include reinforced plastics and the like, which are easily formed and processed.

Note that details regarding the internal configuration of the diffusion promotion device 100 will be described later.

### Configuration of Liquid Agent Supply Device 200

In the embodiment, the liquid agent supply device 200 employs a device configured similarly to the liquid agent applicator disclosed in the prior art 1.

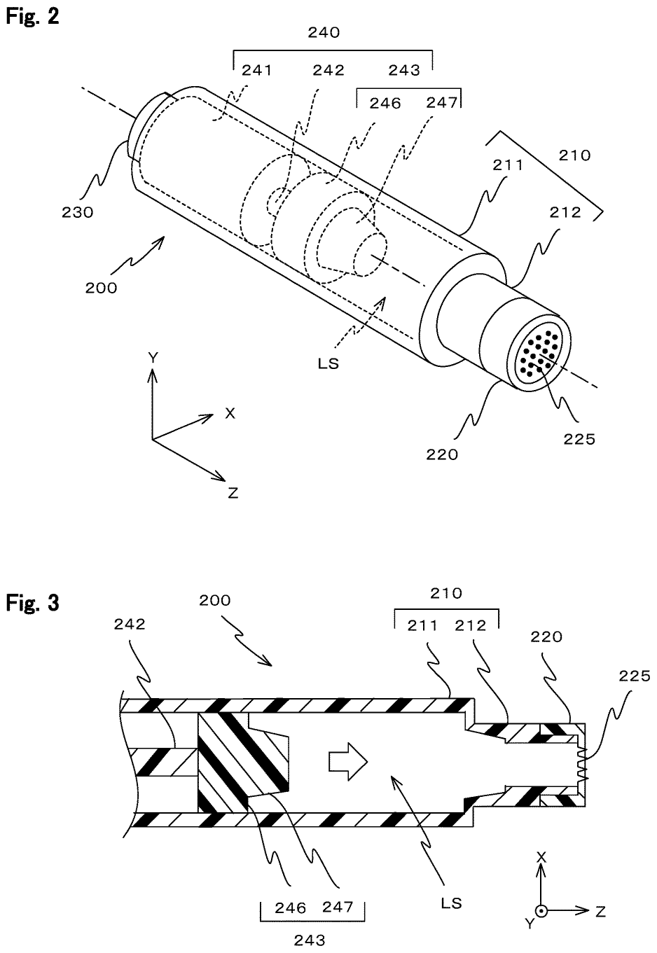

Specifically, the liquid agent supply device 200, as illustrated in FIG. 2, includes a shaft portion 210 and a liquid agent supply unit 220. Furthermore, the liquid agent supply device 200 includes a pressing member (a first pressing operation member) 230.

The shaft portion 210 includes a hollow cylindrical member made of reinforced plastic, at least a portion of which is optically transparent. As illustrated in FIG. 2, the shaft portion 210 includes a large-diameter portion 211 at a side portion in the −Z direction of the shaft portion 210 and a small-diameter portion 212 positioned in the +Z direction relative to the large-diameter portion 211.

A liquid agent supply unit 220, which is detachably connected with the shaft portion 210, is disposed at the end portion, the first end portion in the +Z direction side of the shaft portion 210. The liquid agent supply unit 220 also includes a hollow cylindrical member made of reinforced plastic, at least one portion of which is optically transparent. The internal space of the liquid agent supply unit 220 communicates with the internal space of the shaft portion 210 when in a state where liquid agent supply is possible (see FIG. 3). As described later, a liquid agent storage space LS is formed of a portion of the internal space that is positioned in the +Z direction side of the shaft portion 210 and the internal space of the liquid agent supply unit 220.

Multiple conical microneedles 225 are disposed at the end portion in the +Z direction side (that is, at the end portion sided in the +Z direction) of the liquid agent supply unit 220. At least one of the microneedles, which are hollow microneedles, a through-hole is formed to connect the liquid agent supply unit 220 to the outside. A liquid agent is ejected to the outside via the through-hole.

The knock member 230 is positioned at the end portion in the −Z direction side, namely, at the end portion sided in the −Z direction of the shaft portion 210. Pressing the end portion of the knock member 230 toward the +Z direction causes the knock member 230 to move toward the +Z direction. In response to this movement of the knock member 230 toward the +Z direction, the liquid agent contained in the liquid agent storage space LS is ejected via the through-hole formed in the microneedles 225.

Note that, after the pressing operation toward the +Z direction of the end portion of the knock member 230 disposed in the −Z direction side is completed, the knock member 230 returns to the Z position that is the position before the pressing operation began.

A liquid agent extrusion unit 240 used to eject liquid agent via the through-hole of the microneedles 225 is accommodated in the internal space of the shaft portion 210. The liquid agent extrusion unit 240 includes a quantitative movement portion 241 positioned in the −Z direction of the shaft portion 210, a shaft 242, and a piston 243.

The quantitative movement portion 241 is configured to receive a pressing force resulting from the knock operation applied to the knock member 230. Upon receiving the pressing force, the quantitative movement portion 241 advances the shaft 242 a predetermined distance in the +Z direction, similarly to a conventional knock-type mechanical pencil does, which is disclosed in the prior art 1.

Note that the shaft 242, which advances by a predetermined distance in response to a pressing operation on the knock member 230, keeps the advanced distance even after the pressing operation ends.

The piston 243 is positioned at the end portion in the +Z direction side of the shaft 242. The piston 243 includes a cylindrical main part 246 and a protrusion part 247 disposed in the +Z direction of the main part 246. Here, the protrusion part 247 has a smaller diameter than the main part 246 and a circular truncated cone shape with an outer diameter that gradually decreases in the +Z direction.

Note that the main part 246 includes an elastic material such as synthetic rubber at least in the portion contacting the inner wall of the shaft portion 210.

FIG. 3 is a schematic XZ cross-sectional view illustrating the portion positioned toward the +Z direction of the liquid agent supply device 200. As illustrated in FIG. 3, in the internal space of the shaft portion 210, the piston 243 is movable in the internal space of the large-diameter portion 211. However, once the end portion in the +Z direction side of the main part 246 reaches the end portion of the wall in the +Z direction side of the large-diameter portion 211, it can no longer move further in the +Z direction. The movement of the piston 243 in the +Z direction occurs, as described above, in response to the pressing operation of the knock member 230.

As clearly illustrated in FIG. 3, the liquid agent storage space LS is defined by the side portion in the +Z direction of the piston 243 in the inner space of shaft portion 210 and the inner space of the liquid agent supply unit 220.

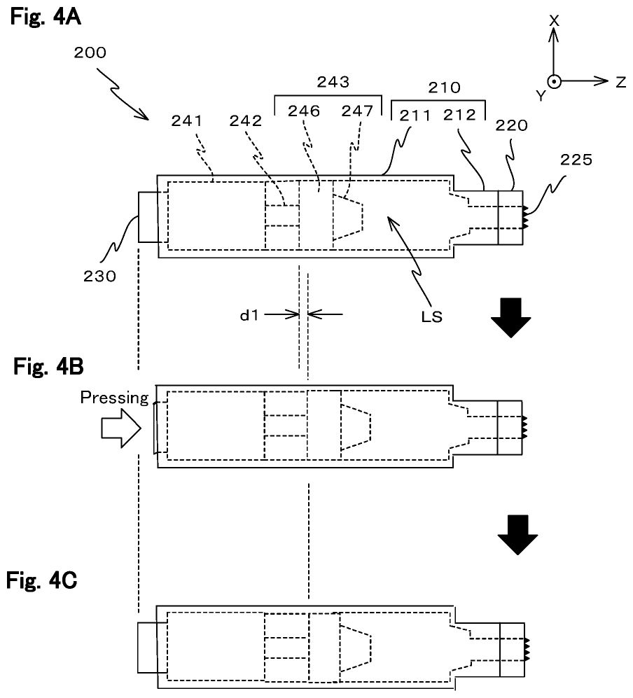

FIGS. 4A, 4B and 4C illustrate the movement of the piston 243 in response to a pressing operation on the knock member 230. Prior to this pressing operation, the piston 243 is at a position in the Z-direction illustrated in FIG. 4A.

When a single pressing operation is performed on the knock member 230 in the state illustrated in FIG. 4A, the shaft 242 moves a predetermined distance d1 in the +Z direction, as illustrated in FIG. 4B. The piston 243, pushed by the shaft 242, then moves the predetermined distance d1 in the +Z direction. As a result, a liquid agent volume corresponding to the predetermined distance d1 in the liquid agent storage space LS is ejected to the outside via the through-hole formed in the microneedles 225.

When the pressing operation on the knock member 230 illustrated in FIG. 4B ceases, the Z position of the knock member 230 returns to the same position as it in FIG. 4A as illustrated in FIG. 4C. Meanwhile, the Z position of the piston 243 remains at the position illustrated in FIG. 4B.

The Liquid Agent Contained in the Liquid Agent Storage space LS

Here, the liquid agent contained in the liquid agent storage space LS is described.

The liquid agent contained in the liquid agent storage space LS includes for example, an aqueous solution containing a powdered culture supernatant of stem cells selected from the group including non-human mammalian dental pulp stem cells, bone marrow stem cells, and adipose stem cells as its main component (an active ingredient). The liquid agent contained in the liquid agent storage space LS is not limited to these examples; aqueous solutions containing the active ingredient with physiological efficacy suitable for the desired cosmetic purpose may be adopted. The stem cells may be those into which a set of specific genes has been introduced by a known method.

Note that, in the embodiment, the active ingredient in the liquid agent is assumed to be negatively ionized.

### Internal Configuration of Diffusion Promotion Device 100

Next, the internal configuration of diffusion promotion device 100 is described.

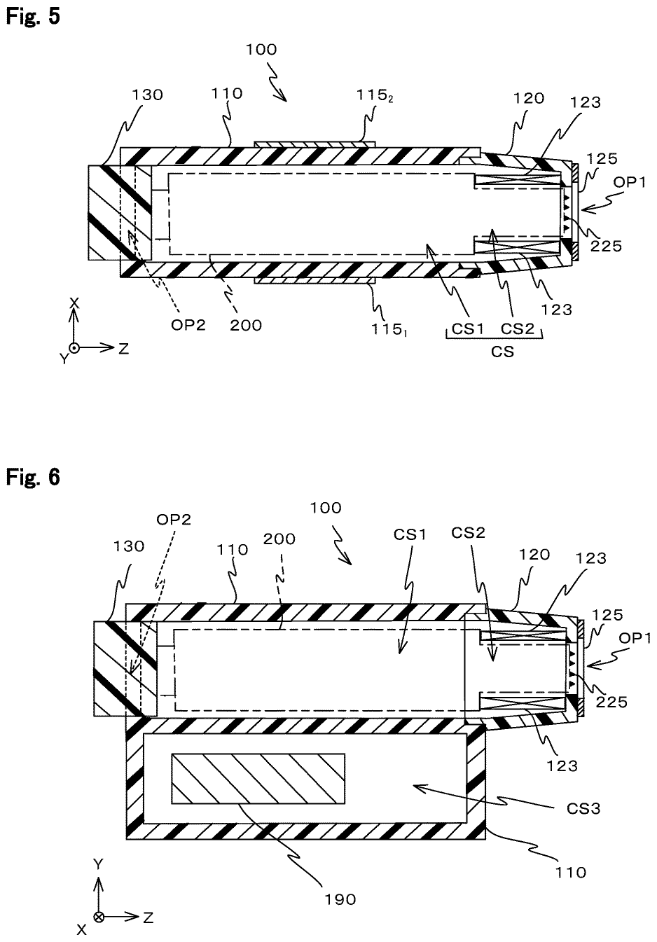

FIGS. 5 and 6 illustrate the internal configuration of the diffusion promotion device 100. Here, FIG. 5 is an XZ cross-sectional view including axis AX (see FIG. 1). FIG. 6 is a YZ cross-sectional view including axis AX.

As illustrated collectively in FIGS. 5 and 6, accommodation spaces CS1 to CS3 are formed inside the diffusion promotion device 100. Here, the accommodation spaces CS1 and CS3 include accommodation spaces defined by the first cylindrical member 110. The accommodation space CS2 includes an accommodation space defined by the second cylindrical member 120.

Both of the accommodation spaces CS1 and CS2 are interconnected each other, and the liquid agent supply device 200 is accommodated in the accommodation space CS formed by the accommodation spaces CS1 and CS2. Furthermore, a battery unit 190 is accommodated in the accommodation space CS3. The accommodation space CS3 is substantially isolated from accommodation space CS due to an internal partition of first cylindrical member 110.

Small holes for electrical wiring, which are not illustrated, are appropriately formed in the internal partition, the outer wall and internal partition of the first cylindrical member 110, and the outer wall of the second cylindrical member 120. Electrical wiring through some of these small holes directly or indirectly makes an electrical connection of an anode of the battery unit 190 with the first electrodes 115₁ and 115₂, and directly or indirectly makes an electrical connection of a cathode of the battery unit 190 with the second electrode 125.

In the embodiment, the battery unit 190 includes a rechargeable battery unit. Electrical wiring between the battery unit 190 and a connector, which is not illustrated, for receiving external electric power used during the charging is routed through some of the small holes.

The second cylindrical member 120 has the first through-hole OP1 formed at the end portion in the −Z direction side thereof, in which the liquid agent supply unit 220 of the liquid agent supply device 200 can pass externally in the Z direction, but the shaft portion 210 cannot pass. Furthermore, the first cylindrical member 110 has the second through-hole OP2 at the end portion in the −Z direction side, which connects the accommodation space CS1 to the exterior and allows the liquid agent supply device 200 to pass through in the Z direction.

The pressing member 130 has a diameter similarly to that of the second through-hole OP2 of the first cylindrical member 110 and is insertable into and removable from the accommodation space CS via the second through-hole OP2. When the portion in the +Z direction side of the pressing member 130 is inserted into the accommodation space CS, the pressing member 130 engages with the inner wall of the first cylindrical member 110 in a manner allowing movement in the accommodation space CS in the Z direction. Here, the pressing member 130 is configured to contact the end surface in the −Z direction side of the knock member 230 of the accommodated liquid agent supply device 200.

The liquid agent supply device 200 is accommodated in the accommodation space CS as follows: with the pressing member 130 removed, the liquid agent supply device 200 may first be inserted into the accommodation space CS in the +Z direction via the second through-hole OP2. Subsequently, the pressing member 130 may be inserted into the accommodation space CS via the second through-hole OP2. Alternatively, the liquid agent supply device 200 may be accommodated in the accommodation space CS as follows. Specifically, with the first cylindrical member 110 and the second cylindrical member 120 separated, the liquid agent supply device 200 may first be inserted into the accommodation space CS1 in the −Z direction, from the end portion in the +Z direction side of the accommodation space CS1. Subsequently, the first cylindrical member 110 and the second cylindrical member 120 may be connected. In the latter case, insertion of the pressing member 130 into the accommodation space CS may be performed at any desired timing.

The elastic member 123 is disposed in the accommodation space CS2. One end portion in the Z direction side of the elastic member 123 contacts or connects to the inner wall surface of the end portion in the +Z direction side of the second cylindrical member 120. The other end portion in the Z direction side of the elastic member 123 contacts the outer wall surface of the end portion in the +Z direction side of the large-diameter portion 211 of the accommodated liquid agent supply device 200. The elastic member 123 biases the accommodated liquid agent supply device 200 in the −Z direction.

The elastic member 123 shortens in the Z direction due to the pressure applied in the +Z direction toward the pressing member 130.

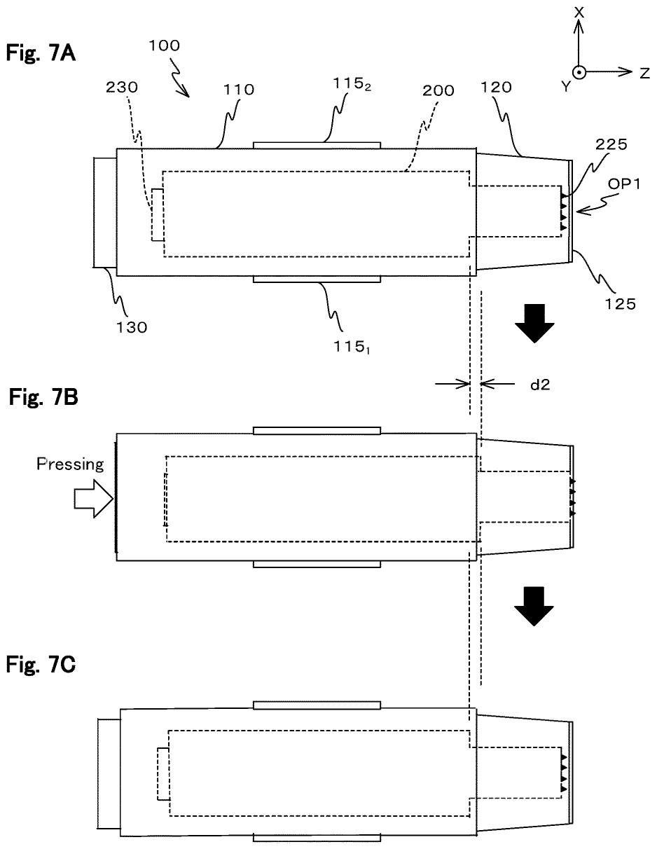

The elastic member 123 shortens in the Z direction until the tip of the microneedles, which has an operating through-hole formed therein, protrudes externally via the first through port OP1, and is configured not to shorten further beyond this point. The elastic member 123 includes, for example, a helical spring and the like. Furthermore, when the liquid agent supply device 200 is accommodated in the accommodation space CS, the elastic member 123 shortens due to the pressure applied in the +Z direction toward the pressing member 130. The amount of shortening of the elastic member 123 in the Z direction is length d2 (see FIGS. 7A, 7B and 7C).

FIGS. 7A, 7B and 7C illustrate illustrates the change in the positional relationship between the diffusion promotion device 100 and the pressing member 130 in response to the pressing operation on the pressing member 130. Prior to the pressing operation, the positional relationship was as illustrated in FIG. 7A.

When the pressing operation is performed once on the pressing member 130 in the state illustrated in FIG. 7A, the liquid agent supply device 200 first moves a distance d2 in the +Z direction relative to the diffusion promotion device 100 as illustrated in FIG. 7B. Subsequently, as the pressing force corresponding to the pressing operation is transmitted to the knock member 230 of the liquid agent supply device 200, the shaft 242 moves a predetermined distance d1 in the +Z direction, as illustrated in FIG. 4B. Then, the piston 243 pushed due to the shaft 242 moves the predetermined distance d1 in the +Z direction. As a result, a liquid agent distance corresponding to the predetermined distance d1 in the liquid agent storage space LS is ejected to the outside via the through-holes formed in the microneedles 225.

During the pressing operation, a current path is formed when the epithelial surface at the site gripping the diffusion promotion device 100 contacts the first electrode 115₁ and/or 115₂, and the second electrode 125 contacts the epithelial surface at the liquid agent application site.

The current path is formed between the first electrode 115₁ and/or 115₂ and the second electrode 125, passing through the layer under the surface of the epithelium of the object. Consequently, a weak current flows through the current path. In response to the generated weak current, the diffusion of the active ingredient contained in the liquid agent is promoted under the epithelium by the so-called ion introduction effect.

When the pressure operation is no longer applied to the pressing member 130 illustrated in FIG. 7B, the knock member 230 returns to its Z-position at the same location indicated in FIG. 4A as illustrated in FIG. 7C. Simultaneously, due to the action of the biasing force in the −Z direction of the elastic member 123, the position of the liquid agent supply device 200 relative to the diffusion promotion device 100 returns to the position illustrated in FIG. 7A. Furthermore, the position of the pressing member 130 in the Z-direction returns to the position illustrated in FIG. 7A.

As described above, the diffusion promotion device 100 of the embodiment, enhances the diffusion of the active ingredient contained in the liquid agent under the surface layer of the object, when the current path is formed between the first electrode 115₁ and/or 115₂ and the second electrode 125, passing through the surface layer of the epithelium of the object, under the condition that the liquid agent is supplied or has been supplied to the object from the liquid agent supply unit 220 of the liquid agent supply device 200, which is accommodated in the internal accommodation space CS. Wherein, the surface layer of the epithelium includes, for example, the skin surface or the stratum corneum. Namely, the diffusion promotion device enhances the diffusion of the liquid agent under the surface layer of the object through the ion introduction effect. Therefore, the diffusion promotion device 100 having a compact configuration enables promotion of diffusion of the active ingredient contained in the supplied liquid agent under the surface layer of the object.

In the embodiment, the active ingredient contained in the liquid agent supplied to the surface layer of the object is efficiently diffused under the surface layer of the object, the liquid agent being supplied by using the microneedles 225 of the liquid agent supply unit 220. Furthermore, even if the liquid agent supplied to the surface layer of the object through the microneedles 225 overflows on the surface layer, the active ingredient contained in the overflowing liquid agent is diffused under the surface layer of the object by the ion introduction effect, when it is percutaneously absorbed by the object such as in the case of the epidermis.

In the embodiment, due to the press of the liquid agent supply unit 220 against the surface layer of the object while pressing the knock member 230 in the +Z direction. By this, the liquid agent is supplied from the tip of the microneedles 225 protruding outside the second cylindrical member 120, via the first through-hole OP1, to the surface layer of the object.

In the embodiment, when the pressure is applied to the pressing member 130, a corresponding pressure force is transmitted to the knock member 230. As a result, the liquid agent is supplied to the surface layer of the object from the tip of the microneedles 225 that protrudes outside the second cylindrical member 120 via the first through-hole OP1. Therefore, through the simple pressing operation on the pressing member 130, the liquid agent can be supplied to the surface layer of the object from the tip of the microneedles 225 protruding outside the second cylindrical member 120 via the first through-hole OP1.

In the embodiment, the elastic member 123 applies a biasing force to accommodate the entire liquid agent supply device 200 in the accommodation space CS when the liquid agent is not being supplied to the object. Therefore, when no pressure is applied to the pressing member 130, the entire liquid agent supply device 200 is automatically accommodated in the accommodation space CS. Consequently, when the liquid agent is not being supplied to the object, the microneedles 225 provided at the end portion in the +Z direction side (that is, at the end portion sided in the +Z direction) of the liquid agent supply unit 220 of the liquid agent supply device 200 is protected without requiring any special operation.

In the embodiment, the first electrodes 115₁ and 115₂ are formed on the surface of the side of the first cylindrical member 110, and the second electrode 125 is formed on the outer surface of the end portion in the +Z direction side of the second cylindrical member 120. Therefore, for example, when an administration target includes a person, the side surface of the diffusion promotion device 100 is held by the person so as to touch the first electrodes 115₁ and/or 115₂ by a palm or at least one finger, for example, 4 fingers other than thumb, or a plurality of fingers, for example, thumb and at least one finger other than that used to press the pressing member 130, and the second electrode 125 is attached onto the epidermis surface of the administration site. By this, the diffusion promotion device 100 enhances the diffusion of the active ingredient under the surface layer of the epithelium at the supply site of the liquid agent.

In the embodiment, the second cylindrical member 120 is detachably connected to the end portion in the +Z direction side (that is, the end portion sided in the +Z direction) of the first cylindrical member 110. Therefore, the liquid agent supply device 200 is accommodated in the accommodation space CS through the following procedure. That is, the liquid agent supply device 200 is first inserted into the first accommodation space CS1 from the end portion in the +Z direction side of the first cylindrical member 110, while the first cylindrical member 110 and the second cylindrical member 120 are separated from each other. Subsequently, the liquid agent supply device 200 is accommodated in the accommodation space CS due to the connection of the first cylindrical member 110 with the second cylindrical member 120.

The ion introduction system 300 of the embodiment includes the diffusion promotion device 100 and the liquid agent supply device 200. Therefore, while the liquid agent is supplied or has been supplied to the object from the liquid agent supply unit 220 of the liquid agent supply device 200, the current path formed between the first electrode 115₁ and/or 115₂ and the second electrode 125, passing through the surface layer of the object enhances the diffusion of the active ingredient contained in the liquid agent under the surface layer of the object through the ion introduction effect. That is, the ion introduction system 300 of the embodiment supplies the liquid agent containing the active ingredient to the object in a compact configuration, and promotes the diffusion of the active ingredient contained in the supplied liquid agent under the surface layer of the object.

### Modifications of the Embodiments

The present invention is not limited to the embodiment and various modifications are possible.

In the embodiment described above, a liquid agent is, for example, supplied to the target object using microneedles. Alternatively, a porous member such as a sponge that absorbs the liquid agent may be used instead of the microneedles. In this case, the liquid agent is supplied to the surface layer of the target object through a simple operation of pressing the porous member, which is saturated with the liquid agent, against the target object. Then, the diffusion of the liquid agent under the surface of the object is promoted by the ion introduction effect.

In the embodiment, the electrical configuration of the diffusion promotion device explicitly specified only the presence of the battery unit. In contrast, the diffusion promotion device includes other elements in addition to the battery unit. Other elements may include a switch element for switching the presence or absence of an electrical connection between the first electrode or the second electrode, a control element for the ON/OFF state of the switch element, a switch instruction input button for instructing the control element to switch the ON/OFF state of the switch element, and an indicator element such as an LED for displaying the ON/OFF state of the switch element.

Additionally, the diffusion promotion device may further include a control element and a display element for detecting and displaying the remaining battery level. It may also further include a control element and a display element for detecting and displaying the remaining liquid level.

Furthermore, in the embodiment, the direct current is assumed to flow between the first electrode and the second electrode. This current may also have a waveform.

In the embodiment, the diffusion promotion device makes a planar surface sided in the +Z-direction of the second electrode. In contrast, the diffusion promotion device may make an uneven-shaped surface sided in the +Z-direction of the second electrode. The uneven-shaped surface may include cases where the protrusions are columnar. Here, the cases where the protrusions are columnar include those where multiple columnar protrusions are formed, creating a comb-like configuration. Adopting the second electrode with such a comb-like shape can, for example, effectively achieve an ion introduction of the active ingredient contained in the liquid agent supplied to the scalp where hair grows.

In the embodiment, since the active ingredient in the liquid agent is negatively ionized, the anode of the battery unit and the first electrode are electrically connected directly or indirectly, and the cathode of the battery unit and the second electrode are electrically connected directly or indirectly. In contrast, when the active ingredient in the liquid agent is positively ionized, the anode of the battery unit and the second electrode are connected electrically, either directly or indirectly, and the cathode of the battery unit and the first electrode are connected electrically, either directly or indirectly.

In the embodiment, the object of the ion introduction is a human. However, the present invention may also be applied to the ion introduction for objects other than human.

### INDUSTRIAL APPLICABILITY

The present invention can be used in the field of the ion introduction for diffusing the active ingredient contained in liquid agents under the surface layer of the object.

### EXPLANATION OF LETTERS OR NUMERALS

100 Diffusion promotion device
110 First cylindrical member
115₁,115₂ First electrode
120 Second cylindrical member
123 Elastic member
125 Second electrode
130 Pressing member (Second pressing operation member)
190 Battery unit
200 Liquid agent supply device
210 Shaft portion
211 Large-diameter portion
212 Small-diameter portion
220 Liquid agent supply unit
225 Microneedle
230 Knock member (First pressing operation member)
240 Liquid agent extrusion unit
241 Quantitative movement portion
242 Shaft
243 Piston
246 Main part
247 Protrusion part
300 Ion introduction system
CS Accommodation space
CS1 First accommodation space (one part of accommodation space CS)
CS2 Second accommodation space (another part of accommodation space CS)
CS3 Third accommodation space
LS Liquid agent storage space
OP1 First through-hole
OP2 Second through-hole

1. A diffusion promotion device having an accommodation space for accommodating a liquid agent supply device for supplying the liquid agent into surface layer of an object is formed internally, which generates an electric current for promoting diffusion of an active ingredient in the liquid agent under a surface of an object, wherein
the liquid agent supply device comprises:
a shaft portion in which a liquid agent storage space for storing the liquid agent is formed along with a first direction; and
a liquid agent supply unit disposed at a first end portion in the first direction side of the shaft portion, supplying the liquid agent contained in the liquid agent storage space to the subject, and
the diffusion promotion device comprises:
a first cylindrical member having a first accommodation space being formed internally for accommodating a predetermined portion located in a second direction side, which is opposite to the first direction side, of the liquid agent supply device, and the first accommodation space is disposed along a third direction parallel to the first direction;
a second cylindrical member having a first through-hole and a second accommodation space being formed internally for accommodating a predetermined portion in the first direction side of the liquid agent supply device and being disposed in the third direction side of the first cylindrical member, wherein the second cylindrical member allows the liquid agent supply device along with the third direction so as to pass the first through-hole, and the liquid agent supply device along with the third direction not so as to pass the first through-hall, and;
a first electrode being used to promote diffusion of the active ingredient;
a second electrode being used to form a current path passing through the surface layer of the object with the first electrode when diffusion of the active ingredient is being promoted; and
a battery unit that generates a predetermined potential difference between the first electrode and the second electrode, and wherein
the accommodation space is formed of the first accommodation space and the second accommodation space.

2. The diffusion promotion device according to claim 1, wherein
the liquid agent supply unit includes a plurality of microneedles disposed at the end portion in the first direction side, each microneedle being hollow to communicate with the liquid agent storage space, and
the diffusion promotion device utilizes the plurality of microneedles to promote diffusion of the active ingredient being contained in the liquid agent supplied into the surface layer of the object.

3. The diffusion promotion device according to claim 2, wherein
the liquid agent supply device includes:
a first pressing operation member disposed at the end portion in the second direction side; and
a liquid agent extrusion unit disposed in the shaft portion, which pushes out the liquid agent in the liquid agent storage space toward the liquid agent supply unit responding to pressure given to the first pressing operation member in the first direction, and wherein
the microneedle is protruded outside the second cylindrical member via the first through-hole in response to the pressure given to the first pressing operation member in the first direction, and then it provides the liquid agent to the object through the tip thereof.

4. The diffusion promotion device according to claim 3 having a second through-hole being formed at the end portion in a fourth direction side of the first cylindrical member through which the shaft portion passes in the third direction, the fourth direction being opposite to the third direction, and further comprising;
a second pressing operation member that is insertable into and removable from the internal space of the first cylindrical member via the second through-hole that transmits a pressing force corresponding to the pressing operation of the second pressing operation member in the third direction to the first pressing operation member of the liquid agent supply device accommodated in the accommodation space.

5. The diffusion promotion device according to claim 3, further comprising
an elastic member, being capable of expanding and contracting in a fourth direction disposed in the second accommodation space, which biases the liquid agent supply device accommodated in the diffusion promotion device in the fourth direction, the fourth direction being opposite to the third direction, wherein
the elastic member applies a biasing force to accommodate the entire liquid agent supply device into the accommodation space when the liquid agent is not supplied to the object.

6. The diffusion promotion device according to claim 1, wherein
the liquid agent supply unit includes a porous member absorbing and retaining the liquid agent, and the unit is disposed at the end portion in the first direction side and conducting, wherein
the porous member provides the liquid agent to the object when it is pressed against the surface of the object, and wherein
the diffusion promotion device promotes the diffusion of the active ingredient contained in the liquid agent provided by pressing the porous member against to the surface of the object under the surface layer of the object.

7. The diffusion promotion device according to claim 1, wherein
the first electrode is formed on at least a portion of the surface of the side of the first cylindrical member.

8. The diffusion promotion device according to claim 1, wherein
the second electrode is formed on at least a portion of the outer surface of the end portion in the third direction side of the second cylindrical member.

9. The diffusion promotion device according to claim 1, wherein
the second cylindrical member is detachably attached to the end portion in the third direction side of the first cylindrical member.

10. An ion introduction system comprising:
the diffusion promotion device according to claim 1; and
the liquid agent supply device, wherein the liquid agent supply device includes:
the shaft portion having the liquid agent storage space to store the liquid agent being formed in the first direction; and
the liquid agent supply unit being disposed at the first end portion in the first direction side of the shaft portion for supplying the liquid agent stored in the liquid agent storage space to the object.
